(19) United States
(12) Patent Application Publication
(10) Pub. No.: US 2007/0276273 A1
(43) Pub. Date: Nov. 29, 2007

(54) Periumbilical Infant Ecg Sensor and Monitoring System

(76) Inventor: Richard L. Watson, Jr, McPherson, KS (US)

Correspondence Address: Mr. Cline H. White;Loeffler Tuggey Pauerstein Rosenthal, 755 East Mulberry Ave., Suite 200, San Antonio, TX, 78212, US

(21) Appl. No.: 10/571,451

(22) PCT Filed: Sep. 9, 2004
(86) PCT No.: PCT/US04/29308

§ 371 Date: Jan. 23, 2007

Related U.S. Application Data
(60) Provisional application No. 60/501,877, filed on Sep. 10, 2003.

(51) Int. Cl. A61B 5/0444

(52) U.S. Cl. 600/511

(57) ABSTRACT

A periumbilical infant ECG sensor for measuring the heartbeat of a newborn infant. The ECG sensor also preferably includes an SAO2 sensor for measuring the oxygen saturation level of the infant's haemoglobin. The ECG sensor is fastenable about the stub of the infant's umbilical cord. The ECG sensor is preferably connected to an electronics module housed in a cavity of an umbilical cord clamp. The electronics module has a power source for supplying power to the ECG sensor and the SAO2 sensor and a transceiver for wirelessly transmitting the ECG and SAO2 signals to a monitoring station.

### BACKGROUND OF THE INVENTION

1. Field of the Invention

The present invention relates generally to electrocardiogram (ECG) and blood oxygenation (SpO₂) sensors, and more particularly to a novel periumbilical sensor array for placement on a newborn infant and its use in association with an umbilical cord clamp, all within a wireless monitoring system.

2. Description of the Related Art

The monitoring of the heart and overall circulatory system of certain newborn infants becomes important when any of a number of symptoms indicate possible congenital or acquired conditions that may require treatment. Two monitoring methods associated with the heart and the circulatory system that are frequently applied to patients of all ages are electrocardiograph measurements and blood oxygenation measurements. Attempts to monitor these parameters for newborn infants meet with specific problems related to the fragility of infant skin surfaces and the tendency of infants to actively reject the placement of wired sensor patches and the like on their bodies.

ECG devices are medical devices that measure the electrical activity of the heart. The beating of the heart is coordinated by electrical nerve impulses and the resultant muscle contractions. This electrical activity of the heart can be measured by an ECG system and the rhythms analyzed to determine if the heart is beating as it should. This measurement and analysis includes the rate and regularity of beats as well as the size and position of the chambers, any damage to the heart, and the effects of drugs or of devices to regulate the heart.

Infants who might have an ECG performed include those with suspected congenital heart disease, cardiomyopathy, or arrhythmia that may have been initially seen on a simple heart rate monitor. The ECG is non-invasive and is typically obtained by placing small pads or electrodes (called leads) on the surface of the infant's chest and abdomen. These leads are attached with wires to the ECG monitoring instrument that reads the electrical signals. The heart rhythm may be displayed and/or printed as a graph to be interpreted by the cardiologist. The ECG trace will indicate what happens to the electrical signal each time the heart beats. The different “waves” or peaks on an ECG trace show the electrical signals within the body triggering different parts of the heartbeat.

ECG measurements can suffer from the introduction of unintended artifacts in the sensed data. Artifacts are changes in the graphed rhythm that are not attributable to the electrical activity of the heart and are common in newborn ECG measurements. Various causes include limb lead reversal, incorrect chest lead positioning, electrical interference from other hospital equipment, and various types of patient movement common in neonates such as hiccoughs.

For most patients, including newborn infants, there are few significant risks associated with the process of obtaining an ECG. The most common is mild irritation of the skin from the application and removal of the electrodes. An ECG monitor is used on most newborn infants in neonatal intensive care units (NICU), such as babies with apnea related to prematurity, or babies receiving medications that elevate the heart rate. However, ECGs are reluctantly carried out for very small premature infants because the chest is so small and the skin is still very fragile.

In a standard twelve-lead ECG, electrodes are attached to the surface of the patient's body, with each electrode corresponding to a particular area of the patient's heart. Because the hearts of neonatal patients are anatomically shifted more to the right of the body than the hearts of adult patients, it is often necessary to monitor neonatal patient's hearts with right-sided chest electrodes for ECG diagnostic and monitoring procedures.

It is often easier to carry out ECG measurements on newborn infants with the much simpler three-lead ECG. While still providing valuable information about the condition of the heart, only three electrodes are positioned on the skin, making for a much easier implementation on small patients. In a three-electrode system, the signal from one electrode is used as a reference signal for a difference between the signals of two other electrodes (e.g. ECG vector). By using this reference signal, and a differential amplifier configuration, a very good (albeit simpler) ECG trace can be acquired.

Accurate placement of the electrodes is a primary concern when preparing a patient for an ECG procedure. If the electrodes are not positioned properly or if they do not properly contact the patient's skin, the recorded data may be invalid. Conventional electrodes are positioned individually on the patient with each electrode being coupled to a separate lead wire. Acquiring a multi-lead ECG for a neonatal patient with conventional electrodes encounters several problems. Accurately positioning and attaching as many as thirteen conventional electrodes to a neonatal patient can be difficult and time consuming even for a skilled clinician. Due to the small chest size of neonatal patients, the conventional electrodes are often too large to fit. Moreover, the electrodes do not adhere well and often irritate the delicate skin of the neonate. The close proximity of the electrodes makes clipping on as many as thirteen lead wires very difficult, with the lead wires often becoming tangled during the attachment process.

Lead wires from electrodes in general will inhibit movement by and around the patient. The wires will stress the electrodes, resulting in malfunction or disconnection from the patient. Because patients need to be moved often during a day, the electrodes also must often be removed and replaced. If not replaced in exactly the same position, the ECG will be different over time. These problems are magnified in the case of neonatal patients whose movements cannot be controlled as easily as the movements of adult patients. Since conventional electrodes do not adhere well to the skin of neonatal patients, the electrodes are even more likely to detach.

Even if the electrodes remain in place and the lead wires remain untangled while a first set of ECG data is acquired; it is difficult to repeat the exact placement of the electrodes in order to acquire subsequent sets of ECG data from the same patient. Consecutive sets of ECG data are often required in order to periodically monitor the patient's recovery progress or general cardiac health. In order to make a valid comparison, the electrode placement for the subsequent sets of ECG data must be the same as the first set of ECG data.

Some efforts have been made to address the problems associated with the placement and use of many electrodes and many lead wires. Wireless ECG systems connect the electrodes to a transmitter to avoid the long wires form the patient to a monitor. Some efforts have also been made to provide an ECG device (both wired and wireless) that eliminates the need to precisely position the electrodes on the patient and prevents the tangling of lead wires. These efforts have generally been directed at providing for a strip of electrodes that are connected to a single bundled lead wire cable. In general, however, these efforts have been unsuccessful in eliminating the problems described above for the neonatal patient. Some of these efforts in the past include the following:

U.S. Pat. No. 6,453,186 B1 issued to Lovejoy et al. on Sep. 17, 2002 entitled Electrocardiogram Electrode Patch describes an electrocardiogram (ECG) electrode patch for attachment to a neonatal or infant patient. The ECG electrode patch includes a plurality of at least three electrodes coupled to a substrate.

U.S. Pat. No. 5,425,362 issued to Siker et al. on Jun. 20, 1995 entitled: Fetal Sensor Device discloses an apparatus and method for non-invasively sensing parameters associated with the health of a fetus, the health of the placenta and the mother. The device includes a probe for inserting the sensors within the uterus of the mother. The sensors are described as potentially measuring heart rate, oxygen saturation, temperature, chemical parameters and electroencephalogram activity.

U.S. Pat. No. 6,551,252 B2 issued to Sackner et al. on Apr. 22, 2003 entitled: Systems and Methods for Ambulatory Monitoring of Physiological Signs describes a system directed to the field of ambulatory monitoring. The sensors include one or more ECG leads and one or more inductive plethysmographic sensors with conductive loops positioned close to the individual to monitor at least basic cardiac parameters, basic pulmonary parameters, or both. The monitoring apparatus also includes a wired unit for receiving data from the sensors, and for storing the data in a computer-readable medium.

U.S. Pat. No. 5,868,671 issued to Mahoney on Feb. 9, 1999 entitled: Multiple ECG Electrode Strip discloses a harness for placement on a patient's chest to allow for ECG measurements. The harness includes a strip of nonconductive film having a connector terminal at one edge for connection to an ECG measuring devise. The strip has a number of electrodes formed on it, each having leads extending to the connector terminal. A backing layer may be peeled-off to expose the electrodes.

U.S. Pat. No. 6,611,705 B2 issued to Hopman et al. on Aug. 26, 2003 entitled Wireless Electrocardiograph System and Method describes a method and system for wireless ECG monitoring. An electrode connector, transmitter and receiver operate with existing electrodes and ECG monitors.

U.S. Patent Application Pub. No. 2004/0073127 A1 filed by Istvan et al. on May 16, 2003 entitled: Wireless ECG System discloses a wireless monitoring system similar to the Hopman et al. system described above. The wireless transceiver is described as being strapped to the arm of the patient and removably connected to the sensor wire leads.

As indicated above, hemoglobin oxygen saturation is a second circulatory system parameter that can provide valuable information about a patient, especially neonatal patients with symptoms of congenital problems. An oxygen saturation monitor in its most common form, known as a pulse oximeter, is a medical device used to monitor the amount of oxygen in the blood. In its most basic form, a small cuff with a light element and a light sensor is wrapped around the infant's foot, hand, toe, or finger. Light passes through the tissues from one side of the cuff to the other. The light waves are altered by the amount of oxygen in the blood, the measurement of which allows for a calculation of the percentage of blood oxygenation. The typical prior art approach for measuring oxygen saturation uses a large non-portable bedside unit, or a portable unit with recording capabilities limited to oxygen saturation. Such devices typically display a measurement in a hospital or laboratory setting. Such devices, when portable, typically are limited to short duration recording or recording only of oxygen saturation.

Pulse oximetry is currently the most widely used, non-invasive form of oxygen monitoring. Some pulse oximeters are built into cardio-respiratory monitors, which also display the heart rate, respiration characteristics, and blood pressure. The oximeter allows the amount of oxygen in the blood to be monitored without having to actually draw blood from the patient for laboratory testing. Pulse oximeters are not typically harmful to infants. They do not require frequent rotation of sample sites and do not burn or require calibration. However, they are subject to false readings and false alarms due to the frequent and uncontrolled movement of the infant patient Some efforts have been made in the past to address the problems associated with pulse oximetry in the neonatal environment. These include the following:

U.S. Pat. No. 6,125,296 issued to Hubelbank on Sep. 26, 2000 entitled Electrocardiographic and Oxygen Saturation Signal Recording describes a portable machine which records electrocardiograph and oxygen saturation data signals in a removable memory device. The information is provided by an oxygen saturation sensor attached to a patient and having lead wires with electrodes attached directly to the patient.

U.S. Pat. No. 6,470,200 B2 issued to Walker et al. on Oct. 22, 2002 entitled Pacifier Pulse Oximeter Sensor describes a sensor structured to be incorporated into an infant pacifier. This system relies on a reflective technology rather than the transilluminescence of most pulse oximeters.

Other efforts have been made to generally improve pulse oximeters by reducing their size and their power requirements. Efforts have also been made to improve accuracy and the range of information provided. Some of these efforts in the past include the following:

U.S. Pat. No. 6,714,804 B2 issued to Al-Ali et al. on Mar. 30, 2004 entitled: Stereo Pulse Oximeter describes a multi-lead, multi-point system that looks at oxygen saturation in various locations in the patient. The system is described as being particularly useful in the management of persistent pulmonary hypertension in neonates.

U.S. Pat. No. 6,697,658 B2 issued to Al-Ali on Feb. 24, 2004 entitled: Low Power Pulse Oximeter describes a modified sampling mechanism that serves to reduce the power consumption of a pulse oximeter.

U.S. Pat. No. 6,496,711 B1 issued to Athan et al. on Dec. 17, 2002 entitled: Pulse Oximeter Probe describes a system that utilizes a light to frequency converter to improve accuracy in a portable pulse oximeter unit.

The Sackner et al. device described above does make an effort to combine ECG and oxygen saturation measurements into a single unit but fails entirely to address the concerns associated with the neonatal patient, operating as it does in the ambulatory field. Placement of the combined sensor system on the newborn infant remains the most difficult problem to overcome. Some efforts have been made in the past to attach various devices, some electronic, to the umbilical cord stub of a newborn, where the stub is established by a clamping device that remains in place for a period of time. Most of these however are directed to simple identification of the child as opposed to the monitoring of ECG and/or oxygen saturation information. Some of these efforts include:

U.S. Pat. No. 5,006,830 issued to Merritt on Apr. 9, 1991 entitled: Method and Device for Deterring the Unauthorized Removal of a Newborn from a Defined Area discloses a method and device with a locking umbilical clamp having an attached identification mark and an attached triggering device. A detection system is triggered upon the removal of the umbilical clamp from a defined area. A wristband is provided, also with an identification mark, for attachment to the wrist of a person authorized to remove the newborn from the defined area.

U.S. Pat. No. 5,440,295 issued to Ciecwisz et al. on Aug. 8, 1995 entitled: Apparatus and Method for Preventing Unauthorized Removal of a Newborn Infant from a Predetermined Area describes an apparatus that includes an electrical transponder detachably securable to an umbilical cord clamping device. When the clamping device is closed the transponder unit also becomes attached to the umbilical cord of the infant. When the infant is discharged from the maternity ward the transponder units can be recycled by removal with the umbilical cord clamp.

U.S. Patent Application Pub. No. 2001/0035824 A1 filed by Fourie et al. on Apr. 18, 2001 entitled: Infant Monitoring and Identification Apparatus describes a system and method for monitoring a controlled area in a maternity ward or other healthcare facility. Access-ways to and from the controlled area are provided with monitoring stations and all infants and mothers are provided with a co-operant pair of monitored devices. The devices have communication means enabling wireless communication of identification data. In the case of the infant this identification device is included in the umbilical cord clamp.

While many attempts have been made in the past to provide ECG and SpO₂ monitoring systems that meet the special needs of a neonate, few if any of the devices accommodate the movement and size of a newborn and provide the convenience of a single device that can monitor both the ECG and the oxygen saturation. The goals of data accuracy and patient comfort are simply not met by any system described in the prior art. It would be desirable therefore to have a system for monitoring ECG and SpO₂ in a newborn infant that provides for repeatable accurate placement of the necessary sensors, comfort to the sensitive skin of the newborn, wireless capabilities to eliminate the presence of long sensor leads, and the use of already existing “attachments” to the newborn for the placement and positioning of the wireless transceiver unit.

### SUMMARY OF THE INVENTION

The above-mentioned difficulties in positioning and attaching conventional electrodes and lead wires to neonatal patients to acquire a multi-lead ECG, mean that multi-lead ECGs are acquired from these patients less often than from adult patients and less often than desired. Thus, there is a need in the field for a device that provides easy, accurate, and consistent attachment of an ECG sensor on a neonatal patient. Accordingly, the present invention provides a novel periumbilical ECG sensor for placement on a newborn infant.

It is an advantage of the present invention to reduce the labor and time associated with the positioning and placement of electrodes on a patient for an ECG procedure. It is another advantage of the invention to ensure consistent placement of these electrodes. It is still another advantage of the invention to minimize the total cost of the materials and equipment for implementing an ECG procedure. Yet another advantage of the invention is the elimination of the need for individual lead wires.

It is still a further advantage of the present invention to improve the reliability and integrity of the acquired ECG data. It is still another advantage of the invention to improve the procedure for acquiring ECGs from neonatal patients.

The present invention also permits the collection of saturated oxygen data from the same monitoring equipment in order to further simplify the number of devices and attachments required to monitor the neonate. Conventional SpO₂ data collection devices are subject to false data due to movement and the present invention provides a means to collect the data with minimal risk of artifacts due to movement. The present invention provides a manner of accurately and securely positioning an SpO₂ sensor on an infant to reduce the acquisition of false data.

It is yet another advantage of the present invention to provide a transceiver for wirelessly transmitting the ECG and SpO₂ signals to a monitoring station for display. Various other features and advantages of the invention are set forth in the following drawings, detailed description, and claims.

### BRIEF DESCRIPTION OF THE DRAWINGS

FIG. 1 is a front view of a sensor constructed in accordance with the present invention.

FIG. 2 is a schematic block diagram of the components of the present invention and their functional relationships.

FIG. 3 is a perspective view showing an application of the system of the present invention in place on an infant in a medical care setting.

FIG. 4 is a detailed view of the implementation of the sensor of the present invention in conjunction with an umbilical cord clamp.

### DETAILED DESCRIPTION OF THE PREFERRED EMBODIMENTS

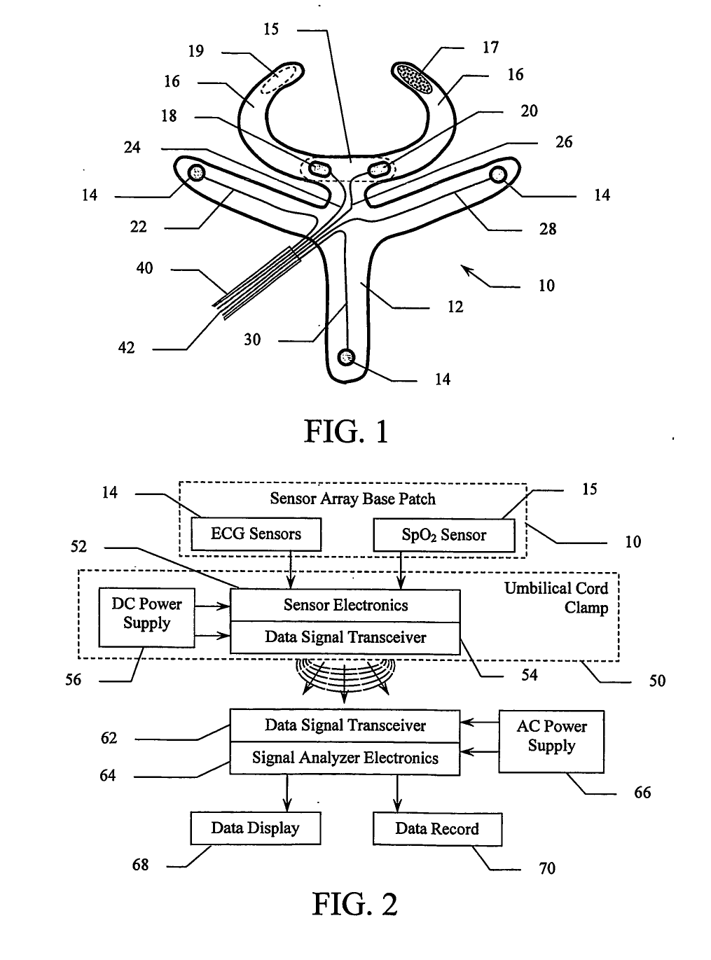

Reference is made first to FIG. 1 for a description of the periumbilical sensor component of the present invention. Periumbilical sensor array 10 in accordance with the present invention preferably comprises a base 12 having three ECG electrodes 14, a hemoglobin oxygen saturation (SpO₂) sensor 15 (comprising a light source 20 and a photo detector 18) and a pair of arms 16 (comprising hook and loop fasteners 17 and 19). ECG electrodes 14 are connected to ribbon cable 40 by way of wires 22, 28, and 30 and are positioned on the distal ends of three radiating extensions of base 12.

Light source 20 and photo detector 18 of SpO₂ sensor 15 are connected to ribbon cable 40 by way of wires 24 and 26. In one preferred embodiment, light source 20 would comprise a visible light LED (light emitting diode) and an infrared LED as is known in the art of pulse oximetry. In such an embodiment, wire 26 may actually comprise two or more wires to provide the necessary electrical connections to drive the light sources of SpO₂ sensor 15. Likewise, wire 24 connected to photo detector 18 may actually comprise two or more wires as necessary to provide the electrical connection to receive the data signal from the photo detector 18, again as is well know in the art of pulse oximetry.

Base 12 is preferably made from a fabric material but may also be made of plastic or other suitable material. Base 12 may be of multi-layer construction to provide both strength (as a substrate for the sensors and wires described above) and comfort for the infant. Adhesive areas, as described in more detail below, may be incorporated into the skin side of base 12 for placement and positioning of the sensor array on the infant.

Reference is now made to FIG. 2 for a description of the various functional components of the present invention and their interaction to provide the monitoring of the ECG and SpO₂ of the infant. FIG. 2 is a schematic block diagram-showing the functional arrangement of the various components including the periumbilical sensor array base patch 10. Sensor array 10 is shown as functionally comprising ECG sensors 14 and SpO₂ sensor 15. These sensors are connected (as described above) to a component incorporated into the enclosure of umbilical cord clamp 50 by way of ribbon cable 40 (shown in FIG. 1). This short length of hardwire connection between the sensor array 10 and the electronic components enclosed within cord clamp 50 is flexible enough and short enough to pose little opportunity for entanglement or discomfort. The actual connection to the cord clamp enclosure is described in more detail below.

The electronic components enclosed in cord clamp 50 enclosure include sensor electronics 52 and data signal transceiver 54. In the preferred embodiment, sensor electronics 52 comprise basic signal processing circuitry necessary to receive and condition the sensor data in a manner that makes the signal suitable for localized RF (radio frequency) transmission. Under some circumstances it may be desirable to utilize non-RF wireless transmissions (such as IR or other frequency light communications) in a manner of substitution well known in the art. Likewise, sensor electronics 52 comprise the basic driver circuitry necessary to drive the light source LED(s) of SpO₂ sensor 15. The specific circuitry associated with the pulse oximetry sensor components is not unique to the present invention and is of the type typically required for transillumination pulse oximetry. Alternate embodiments of the present invention could incorporate more or less of the required circuitry into the cord clamp enclosure 50. In one embodiment, the necessary pulse oximetry sampling controller circuitry could be fully incorporated into the sensor electronics package 52 such that the light source driver circuitry requires no outside input signal to operate. In such an embodiment data signal transceiver 54 may in actuality simply be a transmitter with no requirement that a control signal to the pulse oximetry sensor be transmitted to the unit within the cord clamp enclosure 50.

Finally included within umbilical cord clamp enclosure 50 is DC power supply 56, which in the preferred embodiment is simply a lithium cell battery of sufficient life to supply the sensors and sensor electronics with the necessary voltages typically required for such operation over a number of weeks. Data signal transceiver 54 (which also receives power from DC power supply 56) takes the sensor signal data and provides the necessary electronics (known in the art) to transmit the data signal containing the sensor data in the form of a short range RF transmission. Likewise, in the event the sampling controller components of the pulse oximetry circuitry are not incorporated into sensor electronics 52, signal transceiver 54 will contain the electronic circuitry necessary to receive the RF transmissions that would control the light source drivers for the SpO₂ sensor 15.

At a point remote from the sensor array base patch 10 and the umbilical cord clamp 50 are positioned the necessary electronics to send and receive the data signals to and from the data signal transceiver 54. As described in more detail below, these electronic components would typically be positioned adjacent the bed surface or enclosure that the infant is placed within while monitoring is to occur. Data signal transceiver 62 receives the RF signal from data signal transceiver 54 and pre-processes the signal before passing it to signal analyzer electronics 64. The monitoring base station components 62 and 64 are powered by standard AC power source 66 as is typical in the field of wireless monitoring devices. Also as typical in patient monitoring devices, the signal data may be conditioned and presented for viewing on a data display device 68 (such as an instrument display screen or a chart recorder), or may be sent to a data record storage medium 70 for later retrieval or for distant downloading and review.

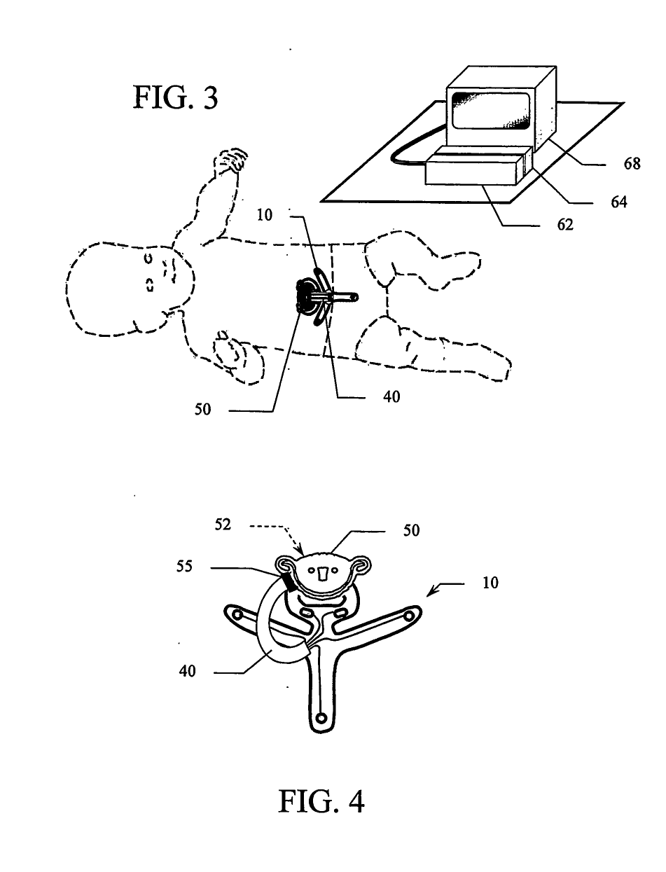

Referring now to FIG. 3, periumbilical sensor array 10 is shown positioned on a newborn infant by fastening arms 16 about the infant's umbilical cord stub. Although arms 16 preferably have hook and loop fasteners 17 and 19 thereon as described above, any suitable fasteners (such as snaps or buckles) will suffice for this purpose. Alternatively, arms 16 may be of sufficient length as to tie them around the umbilical cord stub. The base material of sensor array 10 also preferably has an adhesive, such as an adhesive with a peel-off disposable covering, for firmly adhering the array of electrodes 14 to the skin of the infant. End 42 (shown in FIG. 1) of ribbon cable 40 is connected to the electronics module (not shown) in the rear cavity of an umbilical cord clamp 50 of the type described in U.S. Pat. No. 6,443,958, the disclosure of which is incorporated herein by reference.

Umbilical cord clamp 50 is constructed in a manner that allows it to both cut and seal the umbilical cord after the birth of the infant. The structure is such that once closed the cutting and clamping surfaces are fully enclosed within the clamp which is designed to permanently remain closed even as the umbilical cord stub falls off of the infant. The enclosure associated with this type of clamp permits the incorporation of a small electronics package into its interior with no or only slight modification to the shape and size of the clamp.

As shown in FIG. 3, the infant “wears” the combination of the umbilical cord clamp 50 and the sensor array 10 with the short length of ribbon cable 40 connecting them. The base station monitoring equipment consisting of data signal transceiver 62, signal analyzer electronics 64, and data display device 68, are shown as they would be positioned close to the infant.

FIG. 4 shows in greater detail the arrangement whereby the sensor array 10 of the present invention is connected to the umbilical cord clamp 50 by way of ribbon cable 40. The preferred arrangement shown would comprise a flexible direct connection between the ribbon cable 40 and the individual wires of sensor array 10. Connection of the ribbon cable 40 to the electronics module 52 housed within cord clamp 50 would be removably achieved by way of connector 55 rigidly positioned on cord clamp 50. Other arrangements whereby other ribbon cable connectors may be positioned adjacent the sensor array in place of or in addition to the connector on the cord clamp are anticipated.

As indicated above, the preferred sensor for measuring SpO₂ in the infant's blood hemoglobin is for transilluminational pulse oximetry involving a measurement of oxygenation levels across a capillary bed through interposed skin layers. This can be accomplished at the umbilical cord stub in part because of the highly vascular tissue present at the location and in part because of the manner in which the stub extends away from the infant's skin surface. The light source and light detector components of the pulse oximetry sensor are for clarity shown in FIG. 4 as laying generally in the same plane when in actuality the attachment of arms 16 around the cord stub would place them in an appropriately opposing orientation across the base of the stub. It is anticipated, however, that slight modifications of the sensor arrangement could make it plausible to utilize a reflectance oximetry approach that would not require the sensor components to be positioned in opposition to each other.

Although the foregoing specific details describe a preferred embodiment of this invention, persons reasonably skilled in the art will recognize that various changes may be made in the details of this invention without departing from the spirit and scope of the invention as defined in the appended claims. Therefore, it should be understood that this invention is not to be limited to the specific details shown and described herein.

I claim:

1. A periumbilical ECG sensor device for use on an infant, said device comprising:
a base having at least three electrodes disposed thereon in spaced relation; and
a pair of arms depending from said base, said arms being adaptable for securing said ECG sensor device about the stub of the infant's umbilical cord.

2. The periumbilical ECG sensor device of claim 1 wherein said base comprises at least three radially directed extensions and each of said at least three electrodes is disposed at a distal end of each of said radially directed extensions.

3. The periumbilical ECG sensor device of claim 1 wherein said base further comprises an adhesive layer for attachment of said base to the skin of said infant adjacent said umbilical cord stub.

4. The periumbilical ECG sensor device of claim 1 wherein said pair of arms depending from said base each comprise attachment means for securing one arm to the other after being positioned about the stub of said infant's umbilical cord.

5. The periumbilical ECG sensor device of claim 4 wherein said attachment means comprises a set of hook and loop surfaces.

6. The periumbilical ECG sensor device of claim 1 further comprising an SpO₂ sensor disposed on said base.

7. The periumbilical ECG sensor device of claim 6 wherein said SpO₂ sensor comprises a pulse oximetry device.

8. The periumbilical ECG sensor device of claim 7 wherein said pulse oximetry device comprises a light source and a photo detector, said light source and said photo detector positioned on said pair of arms depending from said base in a manner that places them in opposition across a base of said umbilical cord stub when said arms secure said ECG sensor device about said umbilical cord stub.

9. The periumbilical ECG sensor device of claim 8 wherein said light source comprises a light emitting diode (LED) operable in the visible red and infrared range.

10. A periumbilical ECG sensor system for use on an infant, said system comprising:
a base having at least three electrodes disposed thereon in spaced relation;
a pair of arms depending from said base, said arms being adaptable for securing said ECG sensor device about the stub of the infant's umbilical cord;
an umbilical cord clamp attachable to the stub, said umbilical cord clamp defining an enclosure; and
an electronics module disposed in said enclosure of said clamp, said electronics module comprising a power source and a signal transceiver,
said electrodes being in electrical communication with said electronics module;
wherein said electrodes are operable for generating an ECG signal and said transceiver is operable for transmitting a representation of said ECG signal to a monitoring station.

11. The periumbilical ECG sensor system of claim 10 further comprising an SpO₂ sensor disposed on said base, said SpO₂ sensor being in electrical communication with said electronics module, said SpO₂ sensor being operable for generating an SpO₂ signal representative of the oxygen saturation level of the infant's hemoglobin, and wherein said signal transceiver is further operable for transmitting a representation of said SpO₂ signal to a monitoring station.

12. The periumbilical ECG sensor device of claim 11 wherein said SpO₂ sensor comprises a pulse oximetry device.

13. The periumbilical ECG sensor device of claim 12 wherein said pulse oximetry device comprises a light source and a photo detector, said light source and said photo detector positioned on said pair of arms depending from said base in a manner that places them in opposition across a base of said umbilical cord stub when said arms secure said ECG sensor device about said umbilical cord stub.

14. The periumbilical infant ECG sensor device of claim 13 wherein said light source comprises a light emitting diode (LED) operable in the visible red and infrared range.

15. A periumbilical ECG sensor system for use on an infant, said system comprising:
a base having at least three electrodes disposed thereon in spaced relation, said base further having an adhesive surface for securing said base to the skin of the infant adjacent the stub of the infant's umbilical cord;
an umbilical cord clamp attachable to the stub, said umbilical cord clamp defining an enclosure; and
an electronics module disposed in said enclosure of said clamp, said electronics module comprising a power source and a signal transceiver;
said electrodes being in electrical communication with said electronics module;
wherein said electrodes are operable for generating an ECG signal and said transceiver is operable for transmitting a representation of said ECG signal to a monitoring station.

16. The periumbilical ECG sensor system of claim 15 comprising a removable ribbon cable for providing said electrical communication between said electrodes and said electronics module, said ribbon cable and said electronics module having mating connectors.

17. An infant health monitoring system comprising:
a periumbilical ECG sensor device, said device comprising:
a base having at least three electrodes disposed thereon in spaced relation;
a pair of arms depending from said base, said arms being adaptable for securing said ECG sensor device about the stub of the infant's umbilical cord;
an umbilical cord clamp attachable to the stub, said umbilical cord clamp defining an enclosure;
an electronics module disposed in said enclosure of said clamp, said electronics module comprising a power source and a first signal transceiver, said electrodes being in electrical communication with said electronics module and wherein said electrodes are operable for generating an ECG signal and said first signal transceiver is operable for transmitting a representation of said ECG signal; and
a monitoring station positioned in communication proximity to said first signal transceiver, said monitoring station comprising a second signal transceiver for communication of said representation of said ECG signal.

18. The monitoring system of claim 17 further comprising an SpO₂ sensor disposed on said base, said SpO₂ sensor being in electrical communication with said electronics module, said SpO₂ sensor being operable for generating an SpO₂ signal representative of the oxygen saturation level of the infant's hemoglobin, and wherein said signal transceiver is further operable for transmitting a representation of said SpO₂ signal to a monitoring station.

19. The monitoring system of claim 18 wherein said SpO₂ sensor comprises a pulse oximetry device.

20. The monitoring system of claim 19 wherein said pulse oximetry device comprises a light source and a photo detector, said light source and said light detector positioned on said pair of arms depending from said base in a manner that places them in opposition across a base of said umbilical cord stub when said arms secure said ECG sensor device about said umbilical cord stub.
